(19) United States
(12) Patent Application Publication
(10) Pub. No.: US 2009/0263815 A1
(43) Pub. Date: Oct. 22, 2009

(54) CELL CULTURE SYSTEM, PROCESS FOR THE PRODUCTION THEREOF, AND THE USE THEREOF IN PRECLINICAL INVESTIGATION

(75) Inventor: MANFRED SCHMOLZ, GOMARINGEN (DE)

Correspondence Address: KNOBBE MARTENS OLSON & BEAR LLP, 2040 MAIN STREET, FOURTEENTH FLOOR, IRVINE, CA, 92614, US

(73) Assignee: EDI Experimentelle u. Diagnostische Immunologie GmbH, REUTLINGEN (DE)

(21) Appl. No.: 12/428,355

(22) Filed: Apr. 22, 2009

Related U.S. Application Data
(63) Continuation of application No. PCT/EP2007/009243, filed on Oct. 25, 2007.

(30) Foreign Application Priority Data
Oct. 25, 2006 (DE) 10 2006 051 283.9

(51) Int. Cl. C12Q 1/68; C12M 3/00; C12M 1/24; C12Q 1/02

(52) U.S. Cl. 435/6; 435/289.1; 435/305.2; 435/29

(57) ABSTRACT

The invention relates to a cell culture system, in particular for the preclinical testing of active substances, comprising a first and a second compartment which are in communication with one another via a separating layer between the first and the second compartment, the separation layer being permeable for cellularly secreted substances, wherein the first compartment includes a syntopic culture with tissue cells and immune cells and the second compartment includes a culture with blood cells. The invention further relates to a method in this regard and to a kit and uses for the preclinical testing of active substances.

### CROSS REFERENCE TO RELATED APPLICATIONS

This application is a continuation of copending International Patent Application PCT/EP 2007/009243 filed on Oct. 25, 2007, and designating the US, published in German.

### BACKGROUND OF THE INVENTION

The present invention relates to a cell culture system, a process for the production thereof, a kit, and the use of the cell culture system for preclinical testing of active substances.

Preclinical active substance screening or preclinical testing of active substances is particularly important for the medical validation of substances before they are tested, after successfully passing through this preclinical testing phase, in clinical studies on humans. A further priority of preclinical testing, besides establishing a principal medical effect of the substances to be tested, is also the estimation of possible side effects which might arise in further validation of the active substances in clinical studies.

Thus, for example, early recognition of unwanted side effects makes it possible save costs. In addition, the risk for clinical test patients can be distinctly reduced by a sophisticated preclinical investigation of active substances.

Particularly suitable preclinical investigation models are experimental animals. Animal experiments have the advantage that the active substances to be investigated can be characterized in vivo. However, the information obtained there-from can, because of the in some cases serious differences between animals and humans, be applied to humans to only a limited extent.

Cell cultures are also employed as investigation models in the preclinical phase of testing active substances. The advantage of employing cell cultures is that they can be carried out relatively easily. In addition, cell cultures permit high sample throughput rates and very easily controllable test conditions. In addition, there are no ethical concerns about cell cultures. A disadvantage is that cell cultures can only inadequately simulate, as in vitro investigation models, the cellular processes actually taking place in human tissues.

So-called co-cultures represent an interesting further development of cell cultures. Such co-cultures consist of two cell cultures which are spatially separated from one another but between which exchange of material is possible. One cell culture ordinarily comprises cells of particular tissue types, whereas the other cell culture comprises particular blood cells, especially peripheral blood mononuclear cells (PBMC). For the preclinical testing of active substances, the cell cultures are incubated in the presence of the active substances to be tested. The material fluxes taking place in the co-culture are investigated after the incubation phase and evaluated. For example, such co-cultures are disclosed in the articles “IL-10 producing CD14^{low} monocytes inhibit lymphocyte-dependent activation of intestinal epithelial cells by commensal bacteria” (Haller D, Microbiol. Immunol. 2002; 46: 195-205) and “Monocyte/Macrophage Regulation of Vascular Calcification In Vitro” (Tintut Y, Circulation 2002, 105: 650-655). A disadvantage in this connection is that ultimately co-cultures are also able to reflect the actual circumstances in humans or animals only inadequately, especially in the area of immunoregulatory processes. The information obtained with the aid of such co-cultures must therefore always be regarded with a certain skepticism in relation to a reliable assessment or characterization of the tested active substances. On the other hand, however, the requirements to be met by the quality of a preclinical active substance screening are continually increasing because of the possible clinical risks and of the generally continually increasing costs for developing medicaments.

### SUMMARY OF THE INVENTION

An object of the invention is therefore to provide an in vitro investigation model for preclinical testing of active substances which makes it possible, by comparison with investigation models known from the prior art, to represent better the complex physiological relationships in the human and/or animal body and, in particular, to characterize more reliably the active substances with a view to their clinical investigation.

This object is achieved by a cell culture system, in particular for the preclinical testing of active substances, comprising a first and a second compartment which are in communication with one another via a separating layer, which is permeable for cellularly secreted (excreted) substances, between the first and the second compartment, where the first compartment includes a syntopic culture with tissue cells and immune cells and the second compartment includes a culture with blood cells (blood cell culture).

A syntopic culture in the context of the present invention is intended to mean a cell culture which includes in one compartment at least one tissue cell type and at least one cell type of the immune system.

Whole blood is intended in the context of the present invention to mean blood with all blood constituents, including the blood cells and the blood plasma, and the factors, preferably biologically active factors, present therein, such as, for example, the coagulation factors, complement proteins etc.

Priming is intended to mean in the context of the present invention a preactivation induced by substances, in particular by messengers, of cells.

The invention provides cell culture systems which are distinguished clearly in their cellular complexity from previously disclosed co-culture systems, owing to the taking account of a syntopic culture with tissue cells and immune cells, and of a culture with blood cells. The cell culture system of the invention can be understood to be in particular a syntopic co-culture. Compared with known co-culture systems, the cell culture system of the invention makes it possible for there to be a considerably more complex and in particular more differentiated communication between the cells. The material fluxes and regulatory mechanisms taking place between the cells of the cell culture system permit a distinctly improved simulation of the cellular processes actually taking place in the human and/or animal body. The cellularly excreted substances can react in particular with appropriate target cells in the cell culture system and can for example be reused. It is thus possible particularly advantageously to avoid unnatural excessive concentrations in the cell culture system of the invention. In addition, the target cells change their own production of signal substances under the influence of the messengers excreted by the other cells. The entire regulatory network of the cell culture system of the invention is modified in a very physiological way thereby. The cell culture system of the invention is particularly suitable for preclinical validation of active substances. It is expedient to use for this purpose cells of a species which is to be treated in a later clinical phase with the active substances to be tested. The cell culture system is incubated together with the active substances to be tested. The material fluxes and/or material changes which take place are preferably detected after the incubation phase and can in particular be compared with the material fluxes and/or material changes of a cell culture system which is incubated without active substances. The data and information derived therefrom can be used as basis for a reliable characterization of the investigated active substances. Thus, it is possible in particular to obtain improved information concerning the medical efficacy of the active substances and concerning possible risks, especially with a view to subsequent clinical investigation.

In an embodiment, the tissue cells of the first compartment are adherent. The tissue cells preferably adhere to the surface of the separating layer. The separating layer can be precoated with suitable substances. The substances may be for example proteins, especially extracellular matrix proteins. The separating layer may for example be coated with collagen, laminin, tenascin etc.

The tissue cells of the first compartment are expediently precultured on the surface of the separating layer. The invention provides in particular for the tissue cells to cover at least partly, preferably completely, the separating layer. The tissue cells can cover the separating layer in particular in the form of a layer, preferably as monolayer.

According to another embodiment, the immune cells are phagocytic immune cells, especially monocytes and/or macrophages. The monocytes and macrophages represent an immunoregulatory switching centre within the immune system. They are involved in particular in inflammatory processes in the human and/or animal body. The syntopic culture of the first compartment is preferably a syntopic culture of tissue cells and immune cells.

The immune cells usually accumulate on the tissue cells, preferably with formation of intermolecular adhesions. The accumulation of the immune cells takes place in particular on the basis of receptors located on the cell surface. It is possible by the taking into account of the immune cells in the cell culture system of the invention for inflammatory processes taking place in the human and/or animal body to be simulated distinctly better. The results derived therefrom within the framework of preclinical active substance screening thus make it possible to characterize more reliably the active substances tested with the aid of the cell culture system of the invention.

The tissue cells of the invention preferably constitute cell types which occur in tissues with an inflammatory disorder, especially in tissues with a chronic inflammatory disorder. The tissues may in particular represent organs. The tissue cells may in particular represent cells of one tissue type. In a further embodiment, the syntopic culture may comprise a plurality of tissue cell types. This increases to a particular extent the possibilities of communication and regulation between the cells of the cell culture system of the invention. It is possible in this way to simulate in a particularly effective manner the physiological relationships in the human and/or animal body, especially at the cellular level.

According to a further embodiment, the tissue cells are epithelial cells and/or epitheloid cells. The tissue cells can be epidermal, bronchial and/or intestinal epithelial cells.

In a further embodiment, the tissue cells are endothelial cells, preferably blood vessel endothelial cells. It is further preferred for the tissue cells to be skin cells, especially keratinocytes, fibroblasts and/or synovial cells (called synoviocytes), and/or chondrocytes. Further suitable tissue cells are neural cells and/or muscle cells, especially smooth muscle cells.

According to a further embodiment, the first compartment includes a syntopic culture with muscle cells, especially smooth muscle cells, and immune cells, with the muscle cells preferably being applied to the upper side of the separating layer. Endothelial cells in particular can be applied to the lower side of the separating layer. It is possible in this way to simulate the relationships of natural blood vessels.

In a further embodiment, the cells of the cell culture system of the invention, especially the cells of the syntopic culture (tissue cells and immune cells), originate from cell lines. The cell lines are preferably of human origin.

In a further embodiment, the cells of the cell culture system of the invention originate from tissue samples and/or from samples of body fluids. The samples may be in particular primary isolates, i.e. samples which have been taken from human and/or animal bodies. The tissue samples and/or the samples of body fluids are preferably of human origin. The body fluids may be in particular blood or urine, preferably blood.

The cells of the syntopic culture of the first compartment may originate for example from tissue samples, whereas the cells of the second compartment ordinarily originate from body fluids, preferably from blood.

It is further possible according to the invention to provide for the cell culture system to include both cells from cell lines and cells from tissue samples and/or from samples of body fluids. The compartments of the cell culture system of the invention expediently each comprise either cells from cell lines or cells from tissue samples and/or from samples of body fluids.

The cultured blood cells of the second compartment may be blood cells of one blood cell type. The blood cells are preferably cells of the immune system, especially cells of the peripheral blood. The blood cells may be for example peripheral blood mononuclear cells (PBMC). The blood cells of the second compartment may furthermore include a plurality of blood cell types, in particular lymphocytes, monocytes, macrophages, platelets and/or erythrocytes. The culture of the second compartment is preferably a culture of whole blood (called whole blood culture). All the blood cells occurring in natural blood are usually present in culture in a whole blood culture.

In a further embodiment, the whole blood is of human origin. The whole blood is preferably fresh blood. In a particularly preferred embodiment, the cell culture system of the invention includes exclusively cells of human origin. This makes improved simulation of the physiological relationships in the human body possible. According to a further embodiment the cells of the cell culture system of the invention originate from the same organism, in particular from the same patient.

The blood cell culture of the second compartment is preferably separated into supernatant and sediment. The supernatant usually comprises the blood plasma. The sediment of the whole blood culture comprises in particular the blood cells, for example erythrocytes, platelets and leukocytes.

In a particularly preferred embodiment of the invention, the tissue cells are activated, preferably with inflammatory changes. The tissue cells are in an activated state in particular as a result of activators present in the cell culture system, in particular proinflammatory activators. Examples of suitable activators are antigens or parts thereof, especially epitopes. The activators may also be superantigens. The activators may be of microbial, in particular bacterial, origin. The activators are preferably constituents of bacterial cell walls. The activators may be in particular glycans, preferably peptidoglycans, for example zymosan. Lipopolysaccharides are also suitable activators. The activators may be furthermore toxins, for example endotoxins. Simulation of inflammatory in vivo processes is possible through the activation of the tissue cells. For example, the cellular processes in disorders with an inflammatory course can be simulated in a satisfactory way through the activation of the tissue cells. The disorders which can be simulated with the aid of the cell culture system of the invention may be in particular osteoarthritis, rheumatoid arthritis, Crohn's disease, ulcerative colitis and inflammatory lung disorders.

In a further embodiment, the tissue cells are activated by messengers, in particular by glycosylated proteins and/or peptides, preferably by cytokines. The messengers can be preferably proinflammatory mediators, for example interferons, interleukins and/or tumour necrosis factors (TNF). Thus, the tissue cells can be activated in particular by at least one messenger from the group comprising interferon γ, interleukin-1, interleukin-6 and tumour necrosis factor α (TNFα). The cellular processes in tissues with inflammatory disorders, especially in organs with inflammatory disorders, can be effectively simulated through the activation of the tissue cells.

In a further embodiment, the cellularly secreted substances in the cell culture system of the invention are generally so-called indicators (indicator compounds) of cellular activity, especially messengers. The cellularly secreted substances are preferably cytokines. The cellularly excreted substances are able to diffuse through the separating layer, which is permeable for them, into both compartments of the cell culture system of the invention and react in particular with the cells present therein. The reaction may be based for example on interactions of the secreted substances with surface receptors of the cells present in the respective compartment. This reduces not only the occurrence of unnatural concentrations, especially of unnatural excessive concentrations, of substances in the cell culture system of the invention. On the contrary, secondary effects of the test substances to be tested can also be visualized for the first time in a relevant manner in this way. This is particularly important for investigating the dose-effect relations of the active substances to be tested.

In a further embodiment, the active substances are natural and/or synthetic active substances. The active substances may also be active substance metabolites. The metabolites are produced for example by cellular digestion of the active substances, in particular with the aid of hepatocytes. The cells used for the cellular digestion, especially hepatocytes, may also be a constituent of the cell culture system of the invention. The active substances may also be present in a suitable administration form, for example together with a pharmaceutically suitable carrier. The active substances may for example be ingredients of creams and/or ointments.

The separating layer, which is permeable for the cellularly secreted substances, between the first and the second compartment expediently has apertures, in particular pores, with a diameter between 0.1 and 5 μm, in particular between 0.2 and 0.45 μm. The separating layer may in particular be a constituent of the first or of the second compartment. The separating layer is preferably the base of the first or of the second compartment. The separating layer may also be formed in one piece with the first or the second compartment, in particular with the first compartment. It is particularly preferred according to the invention for the separating layer to be formed as membrane or diaphragm. The compartments of the cell culture system of the invention can be formed from different materials. Suitable and preferred materials are plastics, especially polystyrene or polycarbonate. According to an embodiment, the compartments of the cell culture system of the invention are formed as containers, in particular as cups, chambers or wells. The compartments may also be part of a support which preferably has a plurality of compartments. The support may therefore be for example a plate with wells, in particular a 6-well, 12-well, 24-well or 96-well plate. The support may also be a generally commercially available trans-well system for co-cultures.

In another embodiment, the first compartment is an upper compartment and the second compartment is a lower compartment of the cell culture system.

The present invention further relates to a method for the preclinical testing of active substances using a cell culture system with a first and a second compartment which are in communication with one another via a separating layer, which is permeable for cellularly secreted (excreted) substances, between the first and the second compartment, where the first compartment includes a syntopic culture with tissue cells and immune cells and the second compartment includes a culture with blood cells (blood cell culture), comprising the steps:
addition of the active substances to the cell culture system,
incubation of the cell culture system in the presence of the added active substances,
investigation of the indicators of cellular activity which are detectable in the cell culture system.

In a preferred embodiment, the active substances are added to the syntopic culture of the first compartment. In some cases it may be desired to investigate the influence of active substances on tissues when the active substances pass from the blood circulation into the relevant tissues. It is preferred according to the invention in these cases to add the active substances to the blood cell culture of the second compartment. The physiological blood circulation is simulated by the blood cell culture of the second compartment, and the relevant tissue is simulated by the syntopic culture of the first compartment, in this way.

According to another embodiment, a syntopic culture of tissue cells and immune cells, especially phagocytic immune cells, preferably monocytes and/or macrophages, is used.

In a further embodiment, the cell culture system, in particular the syntopic culture, is subjected to a so-called priming before incubation, preferably before the addition of the active substances. In another embodiment, the priming can also be carried out after the addition of the active substances. It is also possible according to the invention to subject the blood cell culture of the second compartment to a priming. A further possibility is to subject both the syntopic culture and the blood cell culture to a priming. The priming of the syntopic culture and of the blood cell culture can be carried out in particular with respectively different substances. Suitable substances for the priming are in particular mediators or activators, especially inflammatory activators. Concerning further details and features of this, reference is made to the previous description.

According to an embodiment of the invention, the cell culture system is incubated in particular for a period of from 1 to 72 hours, preferably from 2 to 48 hours. The incubation of the cell culture system expediently takes place at a temperature between 20° C. and 40° C., in particular between 35° C. and 40° C., preferably at a temperature of about 37° C. It is possible where appropriate to add further activators during the incubation of the cell culture system.

In a further embodiment of the method of the invention, the investigation of the indicators of cellular activity which are detectable in the cell culture system is undertaken by means of molecular biology methods, in particular at the level of transcription and/or translation, preferably at the post-translational level.

In a further embodiment, the culture medium, in particular in the form of a culture liquid, of the first and/or of the second compartment is investigated to investigate the indicators of cellular activity. Substances secreted by the cells of the cell culture system are preferably investigated. The cellularly secreted substances are investigated in particular by determining the concentrations thereof. Thus, for example, ELISA methods (enzyme linked immunosorbant assay) or electrophoretic methods can be employed for investigating the cellularly secreted substances. It is possible for example to carry out a gel electrophoresis, in particular a two-dimensional polyacrylamide gel electrophoresis (2D PAGE). A further possibility is to use array technologies, in particular multiplex bead arrays. In principle, all biological tests familiar to a person skilled in the art are suitable.

According to yet another embodiment, the cells of the cell culture system are investigated for the investigation of the indicators of cellular activity. It is expedient according to the invention to recover the cells to carry out the investigation, preferably after the incubation of the cell culture system.

The indicators of cellular activity are preferably investigated by investigating cell-associated activation parameters. For example, the calcium influx into the cells of the cell culture system can be measured. The cells can also be investigated for their cAMP/cGAMP level (cyclic adenosine monophosphate/cyclic guanidine-adenosine monophosphate level).

Suitable and preferred cell-associated activation parameters are also the expression of signal transducers and/or receptors in and/or on the cells of the cell culture system. The density of the signal transducers and/or receptors in and/or on the cells is preferably determined. Suitable investigation methods are in particular surface marker analyses, for example histological stains or flow cytometry methods.

A further possibility for investigating cell-associated activation parameters is to investigate the cells of the cell culture system for a change, in particular activation or inhibition, or suppression, of the genes thereof. Northern blot and/or Western blot analyses are suitable for example for constructing profiles of activated genes. Also suitable are planar array technologies, especially gene chips. The genes are preferably investigated on the basis of their gene products. RNA (ribonucleic acid), especially messenger RNA (mRNA), formed by the cells of the cell culture system is preferably investigated. The RNA is expediently isolated from the cells and, in particular, purified. The RNA can be isolated from the cells for example by extraction. For further investigation, the RNA formed is preferably subjected to an amplification, in particular a polymerase chain reaction, preferably with reverse transcriptase (RT-PCR). The actual detection of the RNA is usually undertaken with the aid of a Northern blot technique.

In a further embodiment, proteins expressed by the cells of the cell culture system are investigated to investigate the indicators of cellular activity. Thus, for example, expression patterns or profiles of the proteins can be constructed. It is possible to use in particular mass spectroscopic methods to investigate the proteins. The proteins may be in particular signal transducers and/or receptors. The use of array technologies is likewise possible. The characterization of apoptotic signaling pathways and processes in the cell culture system of the invention as relevant end points is also possible. The methods described in the preceding sections are sufficiently well known to a person skilled in the art, so that a more detailed description can be dispensed with at this point.

According to yet another embodiment of the method of the invention, the indicators of cellular activity are investigated in relation to the indicators of cellular activity of an active substance-free cell culture system, i.e. a cell culture system incubated without active substances. The data or information obtained therefrom allows the investigated active substances to be characterized in more detail. For further details and features, reference is made to the previous description.

The present invention further relates to a kit for the preclinical testing of active substances, which comprises at least one compartment which includes a syntopic culture with tissue cells and immune cells. The immune cells are preferably phagocytic immune cells, in particular monocytes and/or macrophages. The syntopic culture is preferably a syntopic culture of tissue cells and immune cells. The kit of the invention may also comprise a compartment with a culture medium, where the culture medium is preferably suitable for culturing blood cells. The culture medium is in particular a culture liquid, for example a culture solution. The kit of the invention may comprise where appropriate a blood-taking set. For further details and features of the kit, reference is made to the previous description.

The present invention additionally relates to the use of the cell culture system for the preclinical testing of active substances, in particular for investigating dose-effect relations of the active substances. The invention provides in particular for a plurality of cell culture systems to be used, in particular in the form of a parallel approach, for the preclinical validation of the active substances. It is possible in this way for example to test a plurality of active substances in parallel and, in particular, comparatively. It is likewise possible to subject each cell culture system to a different priming. It is further possible to provide according to the invention for the cell culture systems used in a parallel approach to be investigated for different indicators of cellular activity. It is possible in this way to generate overall a larger amount of data concerning the active substances to be investigated, thus making it possible to improve additionally the characterization of the active substances. For further details and features, reference is made to the previous description.

The present invention finally relates to the use of a syntopic culture with tissue cells and immune cells for the preclinical testing of active substances, preferably for investigating dose-effect relations of the active substances. The immune cells are preferably phagocytic immune cells, in particular monocytes and/or macrophages. The syntopic culture is preferably a syntopic culture of the tissue cells and the immune cells.

### DESCRIPTION OF PREFERRED EMBODIMENTS

### Brief Description of the Figures

Further features and details of the invention are evident from the following descriptions of the Figures. It is possible in this connection for the individual features to be implemented each on its own or as a plurality in combination with one another. The Figures are expressly incorporated in the description by reference.

The Figures show diagrammatically:

FIG. 1: cell culture system,

FIG. 2: messenger or mediator synthesis in classical co-culture systems and a cell culture system of the invention,

FIG. 3: influence of diclofenac on the messenger or mediator synthesis in classical co-culture systems and a cell culture system of the invention.

### DESCRIPTION OF THE FIGURES AND PREFERRED EMBODIMENTS

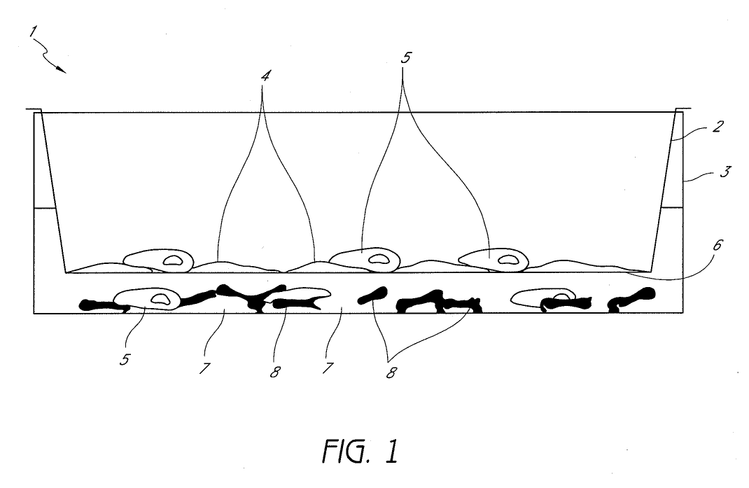

FIG. 1 shows a cell culture system 1 of the invention which consists of an upper container 2 designed as first compartment and of a lower container 3 designed as second compartment. The upper container 2 includes a syntopic culture of synoviocytes 4 and monocytes or macrophages 5. The synoviocytes 4 are adherent and cover in the form of a layer the base 6, which is designed as separating layer, of the upper container 2. The monocytes or macrophages 5 lie on the synoviocytes 4 and are in direct contact with the latter. The synoviocytes 4 and the monocytes or macrophages 5 are derived from cell lines of human origin. The base 6 of the upper container 2 is permeable for cellularly secreted substances. Material exchange between the upper container 2 and the lower container 3 is possible in this way. The lower container 3 includes a whole blood culture of human origin with blood cells 7 and 8. The blood cells are in particular monocytes or macrophages 5, lymphocytes 7 and erythrocytes 8. The whole blood culture is separated into supernatant and sediment. The cells present in the cell culture system 1, and the material fluxes taking place between the cells, make an improved simulation of the corresponding cellular processes in the human body possible. The cell culture system 1 can therefore be used in a particularly suitable manner for preclinical investigation of active substances. The data or information obtained therefrom, especially relating to a principal medical effect, any side effects and a suitable dosage or the active substances, represent a reliable assessment basis for clinical investigation of the active substances.

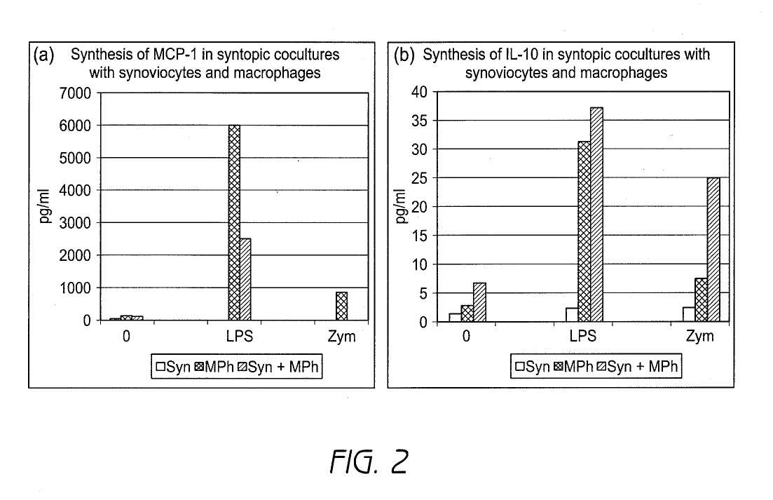

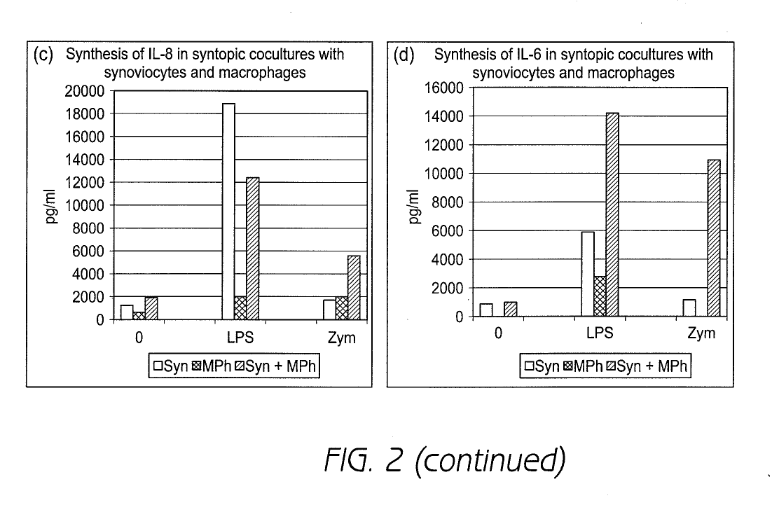

FIG. 2 shows the influence of various stimulants on the synthesis of messengers or mediators, both in classical co-culture systems, and in a cell culture system of the invention. The classical co-culture systems were culture systems which comprised either a culture with synoviocytes (“Syn”) or a culture with macrophages (“MPh”) in their upper compartment, and a culture of whole blood (whole blood culture) in their lower compartment. The cell culture system of the invention comprised a syntopic culture of synoviocytes and macrophages (“Syn+MPh”) in its upper compartment, and a culture of whole blood (whole blood culture) in its lower compartment. The culture systems were exposed to three different stimulatory conditions:
No stimulation (“0”)
Stimulation with a lipopolysaccharide (“LPS”)
Stimulation with zymosan (“Zym”).

The ordinate in FIGS. 2a to 2d shows in each case the amount of the investigated synthesized messenger in picogrammes per milliliter [pg/ml]. The stimulatory conditions are detailed on the abscissa of FIGS. 2a to 2d.

FIGS. 2a to 2d show that different amounts of messengers were synthesized depending on the stimulatory conditions, the measured end points (synthesis of MCP-1 in the case of FIG. 2a, synthesis of IL-10 in the case of FIG. 2b, synthesis of IL-8 in the case of FIG. 2c and synthesis of IL-6 in the case of FIG. 2d), and the culture systems used.

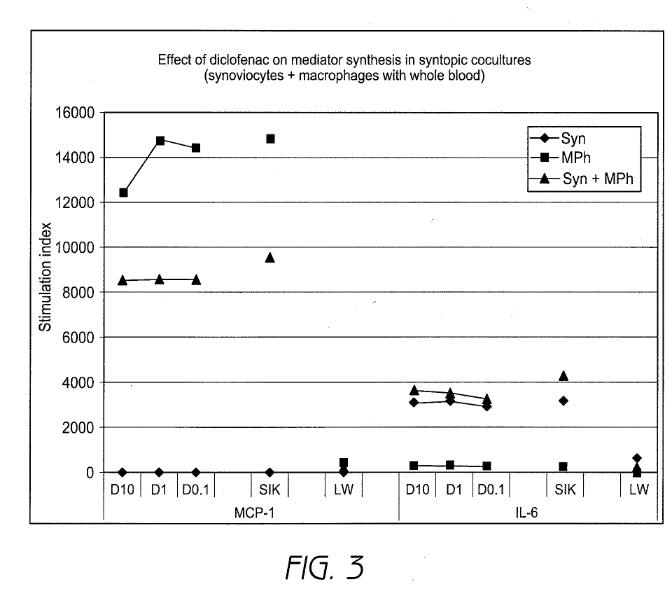

FIG. 3 shows the influence of diclofenac (non-steroidal analgesic) on the messenger and mediator synthesis in classical co-culture systems and a cell culture system of the invention. Concerning the features of the classical co-culture systems and of the cell culture system of the invention, reference is made to the Figure description for FIG. 2. The synthesis of MCP-1 and IL-6 was measured as end points. Activation of the cells by a lipopolysaccharide was chosen as stimulatory condition. The ordinate of FIG. 3 shows the so-called stimulation index. The measured end points are listed on the abscissa of FIG. 3.

The results shown in FIGS. 2 and 3 were carried out by means of a multiplex analysis based on a so-called bead array test with the aid of Luminex(™) technology. For this purpose, colour-coded beads with specific antibodies were employed for binding the messengers. The content of messengers was measured with the aid of a second antibody which was fluorescence-labelled.

Therefore, what is claimed, is:

1. A cell culture system, in particular for the preclinical testing of active substances, comprising a first and a second compartment which are in communication with one another via a separating layer between the first and the second compartment, the separating layer being permeable for cellularly secreted substances, wherein the first compartment includes a syntopic culture with tissue cells and immune cells and the second compartment includes a culture with blood cells.

2. The cell culture system of claim 1, wherein the immune cells are phagocytic immune cells, especially monocytes and/or macrophages.

3. The cell culture system of claim 1, wherein the tissue cells are cells which occur in tissues with inflammatory disorders.

4. The cell culture system of claim 1, wherein the tissue cells are selected from epithelial cells, epitheloid cells, bronchial cells, intestinal epithelial cells, endothelial cells, blood vessel endothelial cells, skin cells, synovial cells (synoviocytes) and/or chondrocytes.

5. The cell culture system of claim 1, wherein the cells of the cell culture system are derived from cell lines, preferably of human origin.

6. The cell culture system of claim 1, wherein the cells of the cell culture system originate from tissue samples and/or from samples of body fluids.

7. The cell culture system of claim 1, wherein the culture of the second compartment is a culture of whole blood (whole blood culture).

8. The cell culture system of claim 1, wherein the tissue cells are activated, in particular have inflammatory changes.

9. The cell culture system of claim 1, wherein the cellularly secreted substances are indicators of cellular activity, especially messengers, preferably cytokines.

10. The cell culture system of claim 1, wherein the active substances to be tested are biological and/or synthetic active substances.

11. The cell culture system of claim 1, wherein the separating layer has apertures, in particular pores, with a diameter between 0.1 and 5 μm, in particular between 0.2 and 0.45 μm.

12. The cell culture system of claim 1, wherein the compartments of the cell culture system are formed as containers, in particular as cups, chambers or wells.

13. A method for the preclinical testing of active substances, comprising the steps:
providing a cell culture system with a first and a second compartment which are in communication with one another via a separating layer between the first and the second compartment, the separating layer being permeable for cellularly secreted substances, wherein the first compartment includes a syntopic culture with tissue cells and immune cells and the second compartment includes a culture with blood cells,
adding said active substances to the cell culture system,
incubating the cell culture system in the presence of said added active substances,
investigating the indicators of cellular activity which are detectable in the cell culture system.

14. The method according to claim 13, wherein the cell culture system, in particular the syntopic culture, is subjected to a priming before incubation, preferably before the addition of the active substances.

15. The method of claim 13, wherein mediators or activators, in particular inflammatory activators, are used for priming the cell culture system.

16. The method of claim 13, wherein investigating of the indicators of cellular activity is undertaken by means of molecular biology methods, in particular at the level of transcription and/or translation, preferably at the post-translational level.

17. The method of claim 13, wherein substances secreted by the cells of the cell culture system are investigated to investigate the indicators of cellular activity.

18. The method of claim 13, wherein the cells of the cell culture system are investigated to investigate the indicators of cellular activity.

19. The method of claim 13, wherein cell-associated activation parameters, in particular the expression of signal transducers and/or receptors in and/or on the cells of the cell culture system, are investigated to investigate the indicators of cellular activity.

20. The method of claim 13, wherein the indicators of cellular activity are investigated in relation to the indicators of cellular activity of an active substance-free cell culture system.

21. A kit for the preclinical testing of active substances, at least comprising a compartment which includes a syntopic culture with tissue cells and immune cells.

22. The kit of claim 22, further comprising a compartment with a culture medium for blood cells.
